SEQUENCE LISTING (56 sequences)

SEQ ID NO: 1 - DNA, 796 bp, Saccharomyces cerevisiae
agcatgtataacaaacactgatttttgttttgagttttaaaagatatccatttactaacattcgaggtgtacaagcacaagttttgcagtgaaaaacattaagaaaaaccaagttatgaatttgggtccaaacagtaagttattgaaagaatacaagtcacaattgatagaattaaatatagaacaatttgaagccggtatcggtttgattttaggtgacgcttatattagatccagagacgaaggtaaaacttactgtatgcaattcgaatggaaaaacaaggcatatatggatcatgtatgcttgttatacgaccaatgggtcttgtcaccacctcataaaaaggaaagagtcaatcacttgggtaacttagttattacctggggtgcccaaacttttaagcaccaagcttttaataagttggcaaatttgtttattgttaacaacaaaaagacaatcccaaacaacttggtagaaaactatttgacccctatgtctttggcttactggttcatggatgacggtggtaaatgggattacaataagaactccacaaataagagtatcgttttgaacactcaatcttttactttcgaagaagttgaatacttggtaaagggtttgagaaataagttccaattgaactgttacgttaaaattaataagaacaagccaattatatacattgattctatgtcatatttgatattctacaatttgatcaaaccttatttgattccacaaatgatgtataagttaccaaacactatctcctccgaaaccttcttgaaatga

SEQ ID NO: 2 - protein, 235 aa, Saccharomyces cerevisiae
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 3 - DNA, 44 bp, Artificial
  Synthetic DNA
ataatatactagtaacgtaaatactagttagtagatgatagttg

SEQ ID NO: 4 - DNA, 25 bp, Artificial
  Synthetic DNA
tgtatggatgggggtaatagaattg

SEQ ID NO: 5 - DNA, 42 bp, Artificial
  Synthetic DNA
acccccatccatacaagcatgtataacaaacactgatttttg

SEQ ID NO: 6 - DNA, 38 bp, Artificial
  Synthetic DNA
tcttaatgtttttcactgcaaaacttgtgcttgtacac

SEQ ID NO: 7 - DNA, 29 bp, Artificial
  Synthetic DNA
tgaaaaacattaagaaaaaccaagttatg

SEQ ID NO: 8 - DNA, 37 bp, Artificial
  Synthetic DNA
gcgtgacataactaatcatttcaagaaggtttcggag

SEQ ID NO: 9 - DNA, 26 bp, Artificial
  Synthetic DNA
ttagttatgtcacgcttacattcacg

SEQ ID NO: 10 - DNA, 59 bp, Artificial
  Synthetic DNA
aattgcccgactcatattaccctgttatccctaagcttgcaaattaaagccttcgagcg

SEQ ID NO: 11 - DNA, 18 bp, Artificial
  Synthetic DNA
atgagtcgggcaattccg

SEQ ID NO: 12 - DNA, 46 bp, Artificial
  Synthetic DNA
ctgggcctccatgtctatcgttaatatttcgtatgtgtattctttg

SEQ ID NO: 13 - DNA, 20 bp, Artificial
  Synthetic DNA
gacatggaggcccagaatac

SEQ ID NO: 14 - DNA, 24 bp, Artificial
  Synthetic DNA
ggttgtttatgttcggatgtgatg

SEQ ID NO: 15 - DNA, 41 bp, Artificial
  Synthetic DNA
cgaacataaacaaccatgggtaaggaaaagactcacgtttc

SEQ ID NO: 16 - DNA, 46 bp, Artificial
  Synthetic DNA
tattgtcagtactgattagaaaaactcatcgagcatcaaatgaaac

SEQ ID NO: 17 - DNA, 35 bp, Artificial
  Synthetic DNA
tcagtactgacaataaaaagattcttgttttcaag

SEQ ID NO: 18 - DNA, 27 bp, Artificial
  Synthetic DNA
cagtatagcgaccagcattcacatacg

SEQ ID NO: 19 - DNA, 48 bp, Artificial
  Synthetic DNA
atagcatacattatacgaagttatcccacacaccatagcttcaaaatg

SEQ ID NO: 20 - DNA, 37 bp, Artificial
  Synthetic DNA
caccgaaatcttcatcccttagattagattgctatgc

SEQ ID NO: 21 - DNA, 41 bp, Artificial
  Synthetic DNA
gctggtcgctatactgcgtgatttacatatactacaagtcg

SEQ ID NO: 22 - DNA, 48 bp, Artificial
  Synthetic DNA
aaaaacataagacaaattaccctgttatccctatgaccggatgaaacc

SEQ ID NO: 23 - DNA, 37 bp, Artificial
  Synthetic DNA
gggataacagggtaatggtacccaattcgccctatag

SEQ ID NO: 24 - DNA, 20 bp, Artificial
  Synthetic DNA
taccgcacagatgcgtaagg

SEQ ID NO: 25 - DNA, 44 bp, Artificial
  Synthetic DNA
ttacgcatctgtgcggtaaggaatcatagtttcatgattttctg

SEQ ID NO: 26 - DNA, 45 bp, Artificial
  Synthetic DNA
caggatgacgcctaaaaagattctctttttttatgatatttgtac

SEQ ID NO: 27 - DNA, 20 bp, Artificial
  Synthetic DNA
ttaggcgtcatcctgtgctc

SEQ ID NO: 28 - DNA, 35 bp, Artificial
  Synthetic DNA
cacactaaattaataatgaagatttcggtgatccc

SEQ ID NO: 29 - DNA, 33 bp, Artificial
  Synthetic DNA
tattaatttagtgtgtgtatttgtgtttgtgtg

SEQ ID NO: 30 - DNA, 40 bp, Artificial
  Synthetic DNA
gcagattgtactgagagtacgacatcgtcgaatatgattc

SEQ ID NO: 31 - DNA, 26 bp, Artificial
  Synthetic DNA
actctcagtacaatctgctctgatgc

SEQ ID NO: 32 - DNA, 38 bp, Artificial
  Synthetic DNA
ttactagtatattatgctccagcttttgttccctttag

SEQ ID NO: 33 - DNA, 48 bp, Artificial
  Synthetic DNA
tcaaggagaaaaaaccagcatgtataacaaacactgatttttgttttg

SEQ ID NO: 34 - DNA, 38 bp, Artificial
  Synthetic DNA
cggcttctaatccgtgctccagcttttgttccctttag

SEQ ID NO: 35 - DNA, 20 bp, Artificial
  Synthetic DNA
acggattagaagccgccgag

SEQ ID NO: 36 - DNA, 29 bp, Artificial
  Synthetic DNA
ggttttttctccttgacgttaaagtatag

SEQ ID NO: 37 - DNA, 52 bp, Artificial
  Synthetic DNA
catgttctttcctgcgttattaagacccactttcacatttaagttgtttttc

SEQ ID NO: 38 - DNA, 45 bp, Artificial
  Synthetic DNA
ctatcactgatagggagattttcacttttctctatcactgatagg

SEQ ID NO: 39 - DNA, 69 bp, Artificial
  Synthetic DNA
tccctatcagtgatagagattgacatccctatcagtgatagagatactgagcacatcagcaggacgcac

SEQ ID NO: 40 - DNA, 69 bp, Artificial
  Synthetic DNA
aagttaaacaaaattatttctagctttctcctctttaatgaattcggtcagtgcgtcctgctgatgtgc

SEQ ID NO: 41 - DNA, 69 bp, Artificial
  Synthetic DNA
gaaataattttgtttaactttaagaaggagatatacataatgaaaaacattaagaaaaaccaagttatg

SEQ ID NO: 42 - DNA, 100 bp, Artificial
  Synthetic DNA
taggccagtacctcccgcttatgtatttactcgtaggatttgcttcgttcgatcagcacaagccttcaaagatgatcatttcaagaaggtttcggaggag

SEQ ID NO: 43 - DNA, 98 bp, Artificial
  Synthetic DNA
gggaggtactggcctagcgtcgtggcccgggagagacagtttagtagtgactcgcggccagttagggataacagggtaatatggcgattgcaattggc

SEQ ID NO: 44 - DNA, 61 bp, Artificial
  Synthetic DNA
caatcacagggcgggaaataagctacaattaacgccaaaaaattatagagtcgcaacggcc

SEQ ID NO: 45 - DNA, 59 bp, Artificial
  Synthetic DNA
tcccgccctgtgattgaggcgggatggtgtccccacagtatgaccgcactaacagaagg

SEQ ID NO: 46 - DNA, 67 bp, Artificial
  Synthetic DNA
catacatttctccacgggacccacagtcgtagatgcgtaaaatcaaccttggtaagtatccaaatcc

SEQ ID NO: 47 - DNA, 60 bp, Artificial
  Synthetic DNA
cgtggagaaatgtatgaaaccctgtatggagagtgattcagtccagccaggacagaaatg

SEQ ID NO: 48 - DNA, 105 bp, Artificial
  Synthetic DNA
ttctcccaagtgtacgatatcacacctagcgccgtgcaaaaaaaaccacgtcaaataatcaaggcgccttgaatgctcgagggttatttgccgactaccttggtg

SEQ ID NO: 49 - DNA, 73 bp, Artificial
  Synthetic DNA
cgtacacttgggagaagtcagatacgattgcggctcagtatgacgatttttgataattatgaagtgtggtttg

SEQ ID NO: 50 - DNA, 75 bp, Artificial
  Synthetic DNA
cggcagtaccggatcctaaagccgattcaagaaaaattaccctgttatccctattagcgacgaaacccgtaatac

SEQ ID NO: 51 - DNA, 61 bp, Artificial
  Synthetic DNA
ggatccggtactgccgacgcactttagaacggccaccgtcctggtccttttcatcacgtgc

SEQ ID NO: 52 - DNA, 24 bp, Artificial
  Synthetic DNA
taacgcaggaaagaacatgtgagc

SEQ ID NO: 53 - DNA, 19 bp, Artificial
  Synthetic DNA
agcggatcctacctgacgc

SEQ ID NO: 54 - DNA, 19 bp, Artificial
  Synthetic DNA
gcgttacataccggatgcg

SEQ ID NO: 55 - DNA, 19 bp, Artificial
  Synthetic DNA
agcggatcctacctgacgc

SEQ ID NO: 56 - DNA, 24 bp, Artificial
  Synthetic DNA
tggacggcagctgatcctgccagg

(19) United States
(12) Patent Application Publication
(10) Pub. No.: US 2024/0002885 A1
(43) Pub. Date: Jan. 4, 2024

(54) PLASMID FOR TRANSFORMATION, METHOD FOR PRODUCING TRANSFORMANT USING THE SAME, AND TRANSFORMATION METHOD

(71) Applicant: TOYOTA JIDOSHA KABUSHIKI KAISHA, Toyota-shi, Aichi-ken (JP)

(72) Inventor: Toru ONISHI, Toyota-shi (JP)

(73) Assignee: TOYOTA JIDOSHA KABUSHIKI KAISHA, Toyota-shi, Aichi-ken (JP)

(21) Appl. No.: 18/255,151

(22) PCT Filed: Dec. 1, 2021
(86) PCT No.: PCT/JP2021/044013

§ 371 Date: May 31, 2023

(30) Foreign Application Priority Data
Dec. 2, 2020 (JP) 2020-200003

(51) Int. Cl. C12N 15/90; C12N 15/81; C12N 15/70; C12N 15/11; C12N 9/22

(52) U.S. Cl. CPC C12N 15/902; C12N 15/81; C12N 15/70; C12N 15/11; C12N 9/22; C12N 2310/20; C12N 2800/80; C12N 2800/10

(57) ABSTRACT

A stable transformant, in which a gene of interest is incorporated into the genome, is simply and efficiently produced. A plasmid for transformation comprises a site into which a gene of interest is to be incorporated, a pair of homologous recombination sequences sandwiching the site, and a pair of endonuclease target sequences sandwiching the pair of homologous recombination sequences.

### TECHNICAL FIELD

The present disclosure relates to a plasmid for transformation that is used upon introduction of a gene of interest into a host, a method for producing a transformant using the plasmid for transformation, and a transformation method using the plasmid for transformation.

### BACKGROUND ART

In general, a technique of introducing a gene of interest into a host cell from the outside is referred to as transformation or gene recombination, and a cell into which the gene of interest is introduced is referred to as a transformant or a recombinant. By efficiently producing such a transformant utilizing a transformation technique, acceleration and/or efficiency of microbial metabolic engineering can be promoted, for example, utilizing a synthetic biological technique. Herein, the synthetic biological technique means a technique of promptly turning a cycle consisting of the designing, construction and learning of a production host. Among others, in synthetic biology of using a yeast or a procaryote as a host, it is important to efficiently construct a host, namely, to efficiently produce a recombinant microorganism.

Transformation using a yeast as a host is broadly classified into a method of using a circular plasmid into which a gene of interest is incorporated, and a method of using a linear vector comprising a gene of interest. It is easy to introduce a gene of interest into a yeast using a circular plasmid, and a transformed yeast can be produced at a high efficiency of approximately 10⁻² (Non Patent Literature 1, NPL1). On the other hand, when a gene of interest is introduced into a yeast using a linear vector, it is necessary to incorporate the gene of interest into the genome according to homologous recombination. Thus, a transformed yeast can be produced only at an efficiency of approximately 10⁻⁶ (NPL2).

As described above, the method of introducing a gene of interest into a yeast using a circular plasmid is highly efficient. However, such a circular plasmid may be detached in some case, and thus, a stable recombinant yeast cannot be produced. On the other hand, in the method of introducing a gene of interest into a yeast using a linear vector, the gene of interest is stably incorporated into the genome. However, as described above, this method is not considered to be highly efficient.

In order to improve the efficiency of introducing a gene of interest into the genome, known is a technique, in which the target sequence of target-specific endonuclease such as homing endonuclease has previously been introduced into a scheduled introduction site in the genome, and then, the double strands at the site have previously been cleaved (NPL1). Moreover, also known is a technique, in which the double strands of a scheduled introduction site in the genome has previously been cleaved by applying a technique of cleaving any given nucleotide sequence, such as CRISPR-Cas9 or TALEN, instead of the target-specific endonuclease (NPL3). Hence, it is possible to improve homologous recombination efficiency to approximately 10⁻² to 10⁻¹ by previously cleaving the double strands at the site into which a gene of interest is to be introduced.

However, in these methods of improving the efficiency of introducing a gene of interest, it has been necessary to previously introduce a nuclease target sequence into a scheduled introduction site in the genome, or it has been necessary to produce guide RNA or the like to the target site. Thus, these methods of improving the efficiency of introducing a gene of interest are complicated, and require various steps, in addition to production of a DNA fragment for homologous recombination containing a gene of interest and the subsequent transformation using the produced DNA fragment.

In addition, Patent Literature 1, PTL1 discloses a plasmid comprising a selective marker having an intron configured to sandwich a homing endonuclease recognition sequence with telomere seed sequences. In the case of the plasmid disclosed in PTL1, as a result of the expression of the homing endonuclease, the circular plasmid can be converted to linear molecules and can be stably present because of the telomere seed sequence at the terminus.

Furthermore, in Escherichia coli of prokaryote, although transformation efficiency using a plasmid is extremely high, genome modification by homologous recombination using a circular or linear vector is extremely inefficient as compared with yeast. Therefore, in order to improve the homologous recombination efficiency, the standard method is to use Escherichia coli into which a plasmid comprising Red recombinase operon, which constitutes a homologous recombination mechanism of λphage, has been introduced in advance, and to introduce a linear vector into the strain. (NPL4). This method is also used for lactic acid bacteria and Corynebacterium (NPL5 and NPL6). However, this method has a problem that the work becomes complicated because it is necessary to perform transformation twice.

### CITATION LIST

### Patent Literature

PTL 1: US 2016/0017344

### Non Patent Literature

NPL 1: Gietz, R. D., et al. “High-efficiency yeast transformation using the LiAc/SS carrier DNA/PEG method.” Nature Protocols. 2 (2007): 31-34.
NPL 2: Storici, F, et al. “Chromosomal site-specific double-strand breaks are efficiently targeted for repair by oligonucleotides in yeast.” Proc. Natl. Acad. Sci. USA. 100 (2003): 14994-14999.
NPL 3: DiCarlo, J. E., et al. “Genome engineering in Saccharomyces cerevisiae using CRISPR-Cas systems.” Nucleic Acids Res. 41 (2013): 4336-4343.
NPL 4: Zhang, Y., et al. “A new logic for DNA engineering using recombination in Escherichia coli.” Nature Genetics 20 (1998): 123-128.
NPL 5: Peng, Y., et al. “Prophage recombinases-mediated genome engineering in Lactobacillus plantarum.” Microb Cell Fact. 14 (2015): 154.
NPL 6: Huang, Y., et al. “Recombineering using RecET in Corynebacterium glutamicum ATCC14067 via a self-excisable cassette.” Sci Rep 7 (2017): 7916.

### SUMMARY OF INVENTION

### Technical Problem

However, all of the aforementioned methods have been problematic in that a stable transformant, in which a gene of interest is incorporated into the genome, cannot be simply and efficiently produced according to the methods. Hence, considering the aforementioned circumstances, the present disclosure provides a plasmid for transformation capable of simply and efficiently producing a stable transformant, in which a gene of interest is incorporated into the genome, a method for producing a transformant using the same, and a transformation method.

### Solution to Problem

The present disclosure that achieves the aforementioned exemplary embodiments includes the following.
(1) A plasmid for transformation, comprising a site into which a gene of interest is to be incorporated, a pair of homologous recombination sequences sandwiching the site, and a pair of endonuclease target sequences sandwiching the pair of homologous recombination sequences.
(2) The plasmid for transformation according to the above (1), further comprising a target-specific endonuclease gene specifically cleaving the double strands of the endonuclease target sequences in an expressible state.
(3) The plasmid for transformation according to the above (2), wherein the target-specific endonuclease gene is a homing endonuclease gene.
(4) The plasmid for transformation according to the above (3), wherein the endonuclease target sequence is specifically recognized by homing endonuclease.
(5) The plasmid for transformation according to the above (2), further comprising an inducible promoter regulating the expression of the target-specific endonuclease gene.
(6) The plasmid for transformation according to any one of the above (1) to (5), which has the gene of interest that is incorporated into the site.
(7) A method for producing a transformant, comprising steps of: introducing the plasmid for transformation according to the above (6) into a host; and selecting a transformant, in which the gene of interest comprised in the plasmid for transformation is incorporated into the genome of the host via the homologous recombination sequences comprised in the plasmid for transformation, and the gene of interest is then expressed therein.
(8) A transformation method comprising a step of introducing the plasmid for transformation according to the above (6) into a host, wherein the gene of interest comprised in the plasmid for transformation is expressed in the host.
(9) The transformation method according to the above (8), wherein the gene of interest is incorporated into the genome of the host via the homologous recombination sequences comprised in the plasmid for transformation.
   The present specification incorporates the disclosure of JP Patent Application No. 2020-200003 based on which the priority of the present application is claimed.

### Advantageous Effects of Invention

By utilizing the plasmid for transformation according to the present disclosure, a transformant, in which a gene of interest is incorporated into a host genome, can be efficiently produced.

Moreover, since the method for producing a transformant according to the present disclosure utilizes the plasmid for transformation according to the present disclosure, a transformant, in which a gene of interest is incorporated into the host genome, can be efficiently produced.

Furthermore, since the transformation method of the present disclosure utilizes the plasmid for transformation according to the present disclosure, excellent transformation efficiency can be achieved upon the production of a transformant, in which a gene of interest is incorporated into the host genome.

### BRIEF DESCRIPTION OF DRAWINGS

FIG. 1 is a configuration diagram schematically showing main parts of the plasmid for transformation according to the present disclosure;

FIG. 2 is a configuration diagram schematically showing one configuration example of the plasmid for transformation according to the present disclosure; and

FIG. 3 is a configuration diagram schematically showing a mechanism of incorporating a gene of interest into a genome, using the plasmid for transformation according to the present disclosure.

### DESCRIPTION OF EMBODIMENTS

Hereinafter, the present disclosure will be described in more detail, using drawings and examples.

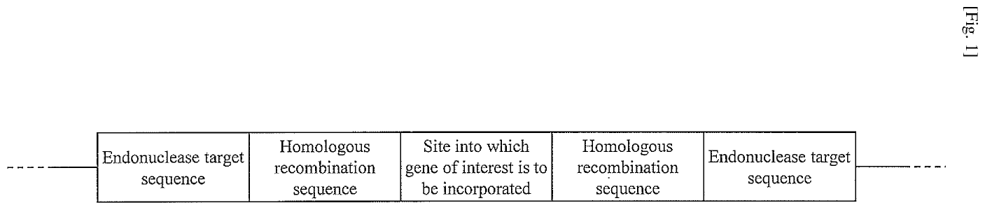

As shown in FIG. 1, the plasmid for transformation according to the present disclosure comprises a site into which a gene of interest is to be incorporated, a pair of homologous recombination sequences sandwiching the site, and a pair of endonuclease target sequences sandwiching the pair of homologous recombination sequences. In other words, having the sense strand of the gene of interest as a reference, the plasmid for transformation comprises, from the 5′-side to the 3′-side, one endonuclease target sequence (which may also be referred to as a “first endonuclease target sequence”), one homologous recombination sequence (which may also be referred to as a “first homologous recombination sequence”), a site into which a gene of interest is to be incorporated, the other homologous recombination sequence (which may also be referred to as a “second homologous recombination sequence”), and the other endonuclease target sequence (which may also be referred to as a “second endonuclease target sequence”) in this order.

The phrase “a site into which a gene of interest is to be incorporated” means a region into which a nucleic acid fragment containing a gene of interest is to be incorporated. Accordingly, such a site into which a gene of interest is to be incorporated is not limited to a specific nucleotide sequence, and can be, for example, one or multiple restriction enzyme target sequences. Moreover, the term “a gene of interest” means a nucleic acid to be introduced into a host genome. Accordingly, such a gene of interest is not limited to a nucleotide sequence encoding a specific protein, and includes nucleic acids consisting of all types of nucleotide sequences, such as a nucleotide sequence encoding siRNA, etc., the nucleotide sequence of a transcriptional regulatory region that regulate the transcription period of a transcriptional product and the production amount thereof, such as a promoter or an enhance, and a nucleotide sequence encoding transfer RNA (tRNA), ribosome RNA (rRNA), etc.

Furthermore, such a gene of interest is incorporated into the above-described site in an expressible state in some embodiments. The term “in an expressible state” means that a gene of interest is linked to a predetermined promoter and is then incorporated into the above-described site, such that the gene of interest can be expressed under the control of the promoter in a host organism.

Further, to such a gene of interest, a promoter and a terminator, and as desired, a cis element such as an enhancer, a splicing signal, a poly A addition signal, a selective marker, a ribosomal binding sequence (SD sequence), and the like can be linked. Examples of the selective marker may include antibiotic resistance genes such as an ampicillin resistance gene, a kanamycin resistance gene, and a hygromycin resistance gene.

The term “a pair of homologous recombination sequences” means a pair of nucleic acid regions having homology to a certain region in a host genome. Such a pair of homologous recombination sequences each cross with the host genome having homology with the homologous recombination sequences, so that a gene of interest sandwiched with the pair of homologous recombination sequences can be incorporated into the host genome. Accordingly, such a pair of homologous recombination sequences are not particularly limited to specific nucleotide sequences, and can be, for example, nucleotide sequences having high homology to the upstream region and downstream region of a certain gene present in the host genome. In this case, if homologous recombination takes place between the plasmid for transformation and the host genome, the gene is deleted from the host genome. As such, the success or failure of homologous recombination can be determined by observing a phenotype caused by the deletion of the gene.

For example, such a pair of homologous recombination sequences can be a region upstream of the coding region of an ADE1 gene associated with an adenine biosynthesis pathway, and a region downstream of the coding region of the ADE1 gene. In this case, if homologous recombination takes place between the pair of homologous recombination sequences and the host genome, an intermediate metabolite of adenine, 5-aminoimidazole riboside is accumulated, and a transformant is colored to red due to the polymerized polyribosylaminoimidazole. Accordingly, by detecting this red color, it can be determined that homologous recombination has taken place between the pair of homologous recombination sequences and the host genome.

Herein, the pair of homologous recombination sequences have high sequence identity to the recombination region in the host genome, to such an extent that they can be homologously recombined (can cross) with one another. The identity between the nucleotide sequences of individual regions can be calculated using conventionally known sequence comparison software “blastn”, etc. The nucleotide sequences of individual regions may have an identity of 60% or more, and the sequence identity is 80% or more in some embodiments, 90% or more in some other embodiments, 95% or more in some other embodiments, and 99% or more in some other embodiments.

Still further, such a pair of homologous recombination sequences may have the same length, or may have each different lengths. The lengths of such a pair of homologous recombination sequences are not particularly limited, as long as the lengths are sufficient for possible homologous recombination (possible crossing). The length of each of the pair of homologous recombination sequences is, for example, 0.1 kb to 3 kb in some embodiments, or 0.5 kb to 3 kb in some embodiments, or 0.5 kb to 2 kb in some other embodiments.

By the way, the plasmid for transformation according to the present disclosure has an endonuclease target sequence outside of the aforementioned pair of homologous recombination sequences (i.e., outside of the aforementioned pair of homologous recombination sequences, when the gene of interest sandwiched by the pair of homologous recombination sequences is defined to be inside). The term “endonuclease target sequence” means a nucleotide sequence recognized by endonuclease.

The endonuclease is not particularly limited, and it broadly means an enzyme having an activity of recognizing a predetermined nucleotide sequence and cleaving double-stranded DNA. Examples of the endonuclease may include restriction enzymes, homing endonuclease, Cas9 nuclease, meganuclease (MN), zinc finger nuclease (ZFN), and transcriptional activation-like effector nuclease (TALEN). Moreover, the term “homing endonuclease” includes both endonuclease encoded by an intron (with the prefix “I-”) and endonuclease included in an intein (with the prefix “PI-”). More specific examples of the homing endonuclease may include I-Ceu I, I-Sce I, I-Onu I, PI-Psp I, and PI-Sce I. Besides, target sequences specifically recognized by these specific endonucleases, namely, endonuclease target sequences are known, and a person skilled in the art could appropriately acquire such endonuclease target sequences.

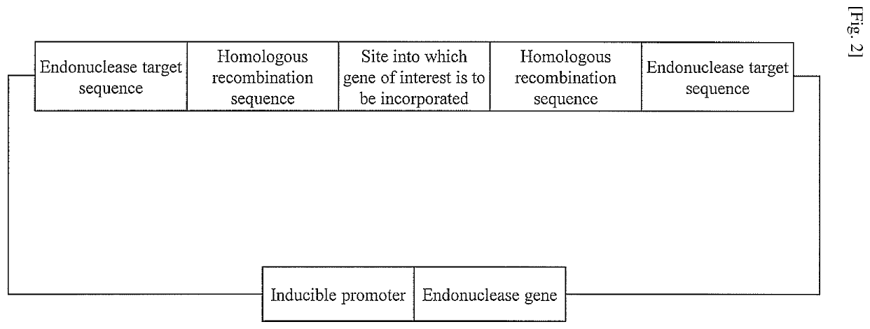

Moreover, as shown in FIG. 2, the plasmid for transformation according to the present disclosure may comprise an inducible promoter and an endonuclease gene. For the expression of an endonuclease gene, not only an inducible promoter, but also a constant expression promoter may be used.

This endonuclease gene encodes an enzyme having an activity of specifically recognizing the aforementioned pair of endonuclease target sequences and cleaving the double strands. That is, examples of the endonuclease gene may include a restriction enzyme gene, a homing endonuclease gene, a Cas9 nuclease gene, a meganuclease gene, a zinc finger nuclease gene, and a transcriptional activation-like effector nuclease gene.

The inducible promoter means a promoter having the function of inducing expression under specific conditions. Examples of the inducible promoter may include, but are not particularly limited to, a promoter inducing expression in the presence of a specific substance, a promoter inducing expression under specific temperature conditions, and a promoter inducing expression in response to various types of stresses. The used promoter can be selected, as appropriate, depending on a host to be transformed.

Examples of the inducible promoter may include galactose inducible promoters such as GAL1 and GAL10, Tet-on/Tet-off system promoters inducing expression by addition or removal of tetracycline or a derivative thereof, and promoters of genes encoding heat shock proteins (HSP) such as HSP10, HSP60 and HSP90. In addition, as such an inducible promoter, a CUP1 promoter that activates by addition of copper ions can also be used. Furthermore, when the host is a prokaryotic cell such as Escherichia coli, examples of the inducible promoter may include a lac promoter inducing expression with IPTG, a cspA promoter inducing expression by cold shock, and an araBAD promoter inducing expression with arabinose.

Further, the method of controlling the expression of an endonuclease gene is not limited to a method of using a promoter such as an inducible promoter or a constant expression promoter, and for example, a method of using DNA recombinase may be applied. An example of the method of turning the expression of a gene ON and OFF may be a FLEx switch method (A FLEX Switch Targets Channelrhodopsin-2 to Multiple Cell Types for Imaging and Long-Range Circuit Mapping. Atasoy et al., The Journal of Neuroscience, 28, 7025-7030, 2008.). According to the FLEx switch method, recombination to change the direction of a promoter sequence is caused by DNA recombinase, so that the expression of a gene can be turned ON and OFF.

On the other hand, the plasmid for transformation according to the present disclosure can be produced based on a conventionally known, available plasmid. Examples of such a plasmid may include: YCp-type Escherichia coli-yeast shuttle vectors, such as pRS413, pRS414, pRS415, pRS416, YCp50, pAUR112, or pAUR123; YEp-type Escherichia coli-yeast shuttle vectors, such as pYES2 or YEp13; YIp-type Escherichia coli-yeast shuttle vectors, such as pRS403, pRS404, pRS405, pRS406, pAUR101, or pAUR135; Escherichia coli-derived plasmids (e.g., ColE-type plasmids, such as pBR322, pBR325, pUC18, pUC19, pUC118, pUC119, pTV118N, pTV119N, pBluescript, pHSG298, pHSG396 or pTrc99A; p15A-type plasmids, such as pACYC177 or pACYC184; pSC101-type plasmids, such as pMW118, pMW119, pMW218 or pMW219; etc.); Agrobacterium-derived plasmids (e.g., pBI101, etc.); and Bacillus subtilis-derived plasmid (e.g., pUB110, pTP5, etc.).

Moreover, the plasmid for transformation according to the present disclosure may further comprise a replication origin, an autonomously replicating sequence (ARS), and a centromere sequence (CEN). The plasmid for transformation comprises these elements, so that it can stably replicate after it has been introduced into a host cell. In addition, the plasmid for transformation according to the present disclosure may comprise a selective marker. The selective marker is not particularly limited, and examples of the selective marker may include a drug resistance marker gene and an auxotrophic marker gene. The plasmid for transformation comprises these selective markers, so that a host cell, into which the plasmid for transformation has been introduced, can be efficiently selected.

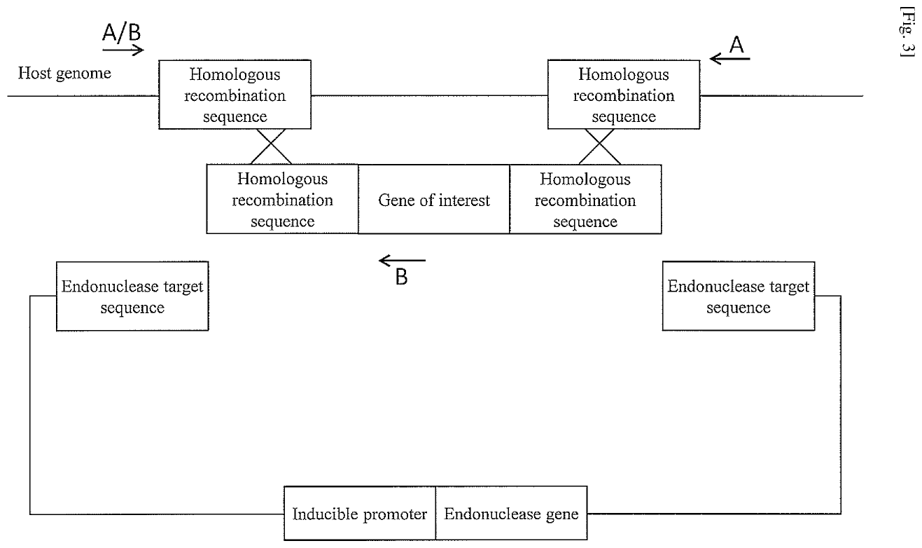

By using the thus configured plasmid for transformation, a stable transformant, in which a gene of interest is incorporated into the genome, can be simply and efficiently produced. To produce a transformant, first, a gene of interest is incorporated into a site, into which such a gene of interest is to be incorporated (FIG. 1). Then, a plasmid for transformation having the gene of interest is introduced into a host cell according to a common method. Thereafter, as schematically shown in FIG. 3, the double stands of a pair of endonuclease target sequences are cleaved by endonuclease that has been expressed under the control of an inducible promoter, so that a nucleic acid fragment containing the gene of interest sandwiched with the pair of homologous recombination sequences is cleaved out. A pair of homologous recombination sequences in the thus cleaved nucleic acid fragment cross with homologous recombination sequences in the host genome, and the gene of interest is then incorporated into the genome. Thereby, a stable transformant, in which the gene of interest is incorporated into the genome, can be produced.

Herein, the method of introducing the plasmid for transformation into which the gene of interest has been incorporated, into a host cell, is not particularly limited, and conventionally known methods such as, for example, a calcium chloride method, a competent cell method, a protoplast or spheroplast method, or an electrical pulse method, can be used, as appropriate. Thereafter, when the plasmid for transformation has a selective marker, the host cell, into the plasmid for transformation has been introduced, can be selected using the selective marker.

Moreover, to allow endonuclease to express under the control of an inducible promoter, conditions are determined, as appropriate, depending on the type of the inducible promoter. For example, when a galactose inducible promoter such as GAL1 or GAL10 is used as such an inducible promoter, galactose is added into a medium for use in the culture of the host cell into which the plasmid for transformation has been introduced, or the host cell is transferred into a galactose-containing medium and is then cultured, so that the expression of the endonuclease can be induced. On the other hand, when a promoter of a gene encoding a heat shock protein (HSP) is used as such an inducible promoter, heat shock is applied, at a desired timing, to the host cell into which the plasmid for transformation has been introduced, upon the culture of the host cell, so that the expression of the endonuclease can be induced at the desired timing.

Furthermore, in the aforementioned plasmid for transformation, when the pair of homologous recombination sequences are set to be nucleotide sequences having high homology to the upstream region and downstream region of a predetermined gene, a fragment containing a gene of interest is incorporated into the genome according to homologous recombination, and at the same time, the predetermined gene is deleted from the genome. Accordingly, by observing a phenotype caused by the deletion of the predetermined gene, whether or not the nucleic acid fragment containing a gene of interest has been incorporated into the genome can be determined. For example, when an ADE1 gene is utilized as such a predetermined gene, if the nucleic acid fragment containing a gene of interest is incorporated into the genome, the ADE1 gene is deleted from the genome. As a result, 5-aminoimidazole riboside is accumulated in the host, and a transformant is colored to red due to the polymerized polyribosylaminoimidazole. Accordingly, by detecting this red color, it can be determined that the nucleic acid fragment containing a gene of interest has been incorporated into the genome of the host.

It is to be noted that, in the aforementioned example, the plasmid for transformation is configured to have an inducible promoter and an endonuclease gene, but that the plasmid for transformation according to the present disclosure may also be configured not to have such an inducible promoter and an endonuclease gene. In this case, an expression vector having an inducible promoter and an endonuclease gene may be prepared, separately, and the expression vector, together with the plasmid for transformation according to the present disclosure, may be introduced into a host cell. Even in this case, in the host cell into which the expression vector having an inducible promoter and an endonuclease gene and the plasmid for transformation having a gene of interest have been introduced, the endonuclease gene is expressed under the control of the inducible promoter, so that, as shown in FIG. 3, a nucleic acid fragment containing a gene of interest sandwiched with a pair of homologous recombination sequences can be cleaved out, and a transformant, in which the gene of interest is incorporated into the genome, can be produced.

Besides, the transformation method using a helper plasmid for transformation according to the present disclosure and the method for producing a transformant are not particularly limited, and these methods can be applied to all types of host cells. Examples of the host cells may include: fungi such as filamentous fungi or yeasts; bacteria such as Escherichia coli or Bacillus subtilis; plant cells; and animal cells including mammals or insects. Among these, yeasts are used as host cells in some embodiments. The type of the yeast is not particularly limited, and examples thereof may include yeasts belonging to genus Saccharomyces, yeasts belonging to genus Kluyveromyces, yeasts belonging to genus Candida, yeasts belonging to genus Pichia, yeasts belonging to genus Schizosaccharomyces, and yeasts belonging to genus Hansenula. More specifically, the aforementioned methods can be applied to yeasts belonging to genus Saccharomyces such as Saccharomyces cerevisiae, Saccharomyces bayanus, or Saccharomyces boulardii.

### EXAMPLES

Hereinafter, the present disclosure will be described in more detail in the following examples. However, these examples are not intended to limit the technical scope of the present disclosure.

### Example 1

In the present example, a monoploid experimental yeast, S. cerevisiae BY4742, was used as a test yeast line.

<Production of Vector to be Introduced into Genome>

The produced two types of vector are YEp-type yeast shuttle vectors, namely, pRS436(SAT)-P_MET25-SCEI-T_CYC1-Sce-5U_ADE1-P_AgTEF1-G418-T_AgTE F1-3U_ADE1-Sce and pRS436(SAT)-P_GAL1-SCEI-T_CYC1-Sce-5U_ADE1-P_AgTEF1-G418-T_AgTEF1-3U_ADE1-Sce (see FIG. 1), each of which is constituted with a sequence formed by inserting S. cerevisiae-derived homing endonuclease I-SceI induced under methionine-deficient conditions or by galactose (SCEI gene; NCBI Accession No. 854590) and a DNA fragment containing a pair of homologous recombination sequences to be introduced into the genome between a pair of I-SceI target sequences (endonuclease target sequences).

Regarding the vector pRS436(SAT)-P_MET25-SCEI-T_CYC1-Sce-5U_ADE1-P_AgTEF1-G418-T_AgTE F1-3U_ADE1-Sce, a SCEI gene to which a MET25 promoter and a CYC1 terminator had been added (a sequence into which the intron of a COX5B gene had been inserted, and in which codons in the whole length had been converted depending on the codon use frequency in the nuclear genome of the yeast; SEQ ID NOS: 1 and 2); a gene sequence containing a nourseothricin resistance gene (nat marker); as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1); and as a marker gene for homologous recombination, a gene sequence containing a G418 resistance gene (G418 marker), to which Ashbya gossypii-derived TEF1 promoter and TEF1 terminator had been added, were inserted into a vector prepared by removing a URA3 gene, a TDH3 promoter, and a CYC1 terminator from the pRS436GAP vector (NCBI Accession No. AB304862). Besides, 5U_ADE1, 3U_ADE1, and the G418 marker were inserted between two homing endonuclease I-SceI target sequences, and could be cleaved by the SCEI gene added to the MET25 promoter that was induced in a methionine-free medium. The sequence cleaved in the yeast cell is introduced into the genome according to homologous recombination (see FIG. 3).

Individual DNA sequences can be amplified by PCR. To bind individual DNA fragments to each other, there were synthesized primers, to each of which a DNA sequence was added to overlap with a DNA sequence adjacent thereto by approximately 15 bp (Table 1). Using these primers, a DNA fragment of interest was amplified with the genome of the S. cerevisiae OC-2 strain or synthetic DNA used as a template, and the DNA fragments were successively bound to each other, using In-Fusion HD Cloning Kit, etc. The resultant was cloned into the pRS436GAP vector to produce a final plasmid of interest.

Regarding pRS436(SAT)-P_GAL1-SCEI-T_CYC1-Sce-5U_ADE1-P_AgTEF1-G418-T_AgTEF1-3U_ADE1-Sce, a SCEI gene to which a GAL1 promoter had been added instead of a MET25 promoter was inserted into the vector. The SCEI gene can be expressed in a medium containing galactose as a carbon source, and a sequence inserted between I-SceI target sequences can be cleaved. The present vector was produced by amplifying a DNA fragment of interest, using pRS436(SAT)-P_MET25-SCEI-T_CYC1-Sce-5U_ADE1-P_AgTEF1-G418-T_AgTE F1-3U_ADE1-Sce or the genome of the S. cerevisiae OC-2 strain as a template (the used primers are shown in Table 1), and then binding the DNA fragments to each other, using In-Fusion HD Cloning Kit, etc.

TABLE 1

| | | |
|---|---|---|
| | | SEQ ID |
| Amplified DNA fragment | Primer sequence (5′-3′) | NO: |

pRS436(SAT)-P_MET25-SCEI-T_CYC1-Sce-5U_ADE1-P_AgTEF1-G418-T_AgTEF1-3U_ADE1-Sce

| | | |
|---|---|---|
| MET25 promoter | ATAATATACTAGTAACGTAAATACTAGTTAGTAGATGAT | 3 |
| | AGTTG | |
| | TGTATGGATGGGGGTAATAGAATTG | 4 |
| COX5B intron | ACCCCCATCCATACAAGCATGTATAACAAACACTGATTTT | 5 |
| | TG | |
| | TCTTAATGTTTTTCACTGCAAAACTTGTGCTTGTACAC | 6 |
| SCEI | TGAAAAACATTAAGAAAAACCAAGTTATG | 7 |
| | GCGTGACATAACTAATCATTTCAAGAAGGTTTCGGAG | 8 |
| CYC1 terminator | TTAGTTATGTCACGCTTACATTCACG | 9 |
| (including I-SceI target | AATTGCCCGACTCATATTACCCTGTTATCCCTAAGCTTGC | 10 |
| sequence) | AAATTAAAGCCTTCGAGCG | |
| 5U_ADE1 | ATGAGTCGGGCAATTCCG | 11 |
| | CTGGGCCTCCATGTCTATCGTTAATATTTCGTATGTGTAT | 12 |
| | TCTTTG | |
| TEF1 promoter derived | GACATGGAGGCCCAGAATAC | 13 |
| from Ashbya gossypli | GGTTGTTTATGTTCGGATGTGATG | 14 |
| G418 | CGAACATAAACAACCATGGGTAAGGAAAAGACTCACGTT | 15 |
| | TC | |
| | TATTGTCAGTACTGATTAGAAAAACTCATCGAGCATCAA | 16 |
| | ATGAAAC | |
| TEF1 terminator derived | TCAGTACTGACAATAAAAAGATTCTTGTTTTCAAG | 17 |
| from Ashbya gossypli | CAGTATAGCGACCAGCATTCACATACG | 18 |
| TEF1 promoter | ATAGCATACATTATACGAAGTTATCCCACACACCATAGC | 19 |
| (including portion of LoxP | TTCAAAATG | |
| sequence) | CACCGAAATCTTCATCCCTTAGATTAGATTGCTATGC | 20 |
| 3U_ADE1 | GCTGGTCGCTATACTGCGTGATTTACATATACTACAAGTC | 21 |
| (including I-SceI target | G | |
| sequence) | AAAAACATAAGACAAATTACCCTGTTATCCCTATGACCG | 22 |
| | GATGAAACC | |
| pRS436 | GGGATAACAGGGTAATGGTACCCAATTCGCCCTATAG | 23 |
| (including 2 μ replication | TACCGCACAGATGCGTAAGG | 24 |
| origin) | | |
| LEU2 terminator | TTACGCATCTGTGCGGTAAGGAATCATAGTTTCATGATTT | 25 |
| | TCTG | |
| | CAGGATGACGCCTAAAAAGATTCTCTTTTTTTATGATATT | 26 |
| | TGTAC | |
| nourseothricin resistance | TTAGGCGTCATCCTGTGCTC | 27 |
| gene | CACACTAAATTAATAATGAAGATTTCGGTGATCCC | 28 |
| CYC1 promoter | TATTAATTTAGTGTGTGTATTTGTGTTTGTGTG | 29 |
| | GCAGATTGTACTGAGAGTACGACATCGTCGAATATGATT | 30 |
| | C | |
| pRS436 | ACTCTCAGTACAATCTGCTCTGATGC | 31 |
| (including ampicillin | TTACTAGTATATTATGCTCCAGCTTTTGTTCCCTTTAG | 32 |
| resistance gene and ColE1 | | |
| replication origin) | | |

pRS436(SAT)-P_GAL1-SCEI-T_CYC1-Sce-5U_ADE1-P_AgTEF1-G418-T_AgTEF1-3U_ADE1-Sce

| | | |
|---|---|---|
| Sequence other than GAL1 | TCAAGGAGAAAAAACCAGCATGTATAACAAACACTGATT | 33 |
| promoter | TTTGTTTTG | |
| | CGGCTTCTAATCCGTGCTCCAGCTTTTGTTCCCTTTAG | 34 |
| GAL1 promoter | ACGGATTAGAAGCCGCCGAG | 35 |
| | GGTTTTTTCTCCTTGACGTTAAAGTATAG | 36 |

<Production of Genome Introduction Vector-Introduced Strain Used for ADE1 Gene Locus>

Using the produced pRS436(SAT)-P_MET25-SCEI-T_CYC1-Sce-5U_ADE1-P_AgTEF1-G418-T_AgTE F1-3U_ADE1-Sce and pRS436(SAT)-P_GAL1-SCEI-T_CYC1-Sce-5U_ADE1-P_AgTEF1-G418-T_AgTEF1-3U_ADE1-Sce, the S. cerevisiae BY4742 strain was transformed, and was then applied to a YPD agar medium containing nourseothricin. Then, growing colonies were purified. Transformation was carried out according to the method of Akada et al. {Akada, R. et al. “Elevated temperature greatly improves transformation of fresh and frozen competent cells in yeast” BioTechniques 28 (2000): 854-856}. The obtained transformants were referred to as the strains “Uz3201” and “Uz3255” strains, respectively.

<Cleaving of DNA Sequence for Homologous Recombination from Genome Introduction Vector and Introduction of the DNA Sequence into ADE1 Gene Locus>

The Uz3201 strain was cultured in a YPD liquid medium supplemented with 75 μg/ml nourseothricin for 1 day, and the obtained culture was then applied to a G418-containing SDC-Met agar medium (a methionine-free, complete synthetic medium) or a YPD medium. In the case of the methionine-free medium, it is considered that homing endonuclease I-SceI is induced, and that the cleaved DNA fragment for homologous recombination has homologous recombination in the ADE1 gene locus, so that the ADE1 gene is disrupted. The ADE1 gene is a gene of adenine biosynthesis pathway, and in the ADE1 gene-disrupted strain, 5-aminoimidazole riboside as an intermediate metabolite of adenine is accumulated, and the polymerized polyribosylaminoimidazole is colored to red. Hence, the ADE1 gene-disrupted strain can be easily distinguished.

The Uz3255 strain was cultured in a YPD liquid medium containing 75 μg/ml nourseothricin for 1 day, and was then applied to a G418-containing YPGa agar medium (carbon source: 2% galactose) or a YPD agar medium. In the YPGa medium, homing endonuclease I-SceI is induced.

Besides, the efficiency of homologous recombination into the ADE1 gene locus was calculated according to the following equations.

ADE1 gene disruption efficiency (%) of Uz3201 strain=Number of red colonies growing in G418-containing SDC-Met agar medium/Number of colonies growing in YPD medium

ADE1 gene disruption efficiency (%) of Uz3255 strain=Number of red colonies growing in G418-containing YPGa agar medium/Number of colonies growing in YPD medium

<Results and Discussion>

The results obtained by studying the efficiency of introducing a nucleic acid fragment into a genome, using two types of plasmids involving different endonuclease induction conditions, are shown in Tables 2 and 3. Table 2 shows the results obtained using a plasmid that induces I-SceI under methionine-free conditions. Table 3 shows the results obtained using a plasmid that induces I-SceI when the carbon source is galactose.

TABLE 2

| | I-SceI induction | I-SceI non-induction |
|---|---|---|
| | conditions | conditions |
| | (SDC-Met + G418) | (YPD + G418) |
| ADE1 gene disruption | 1.57 | 0.00 |
| efficiency (%) | | |

TABLE 3

| | I-SceI induction | I-SceI non-induction |
|---|---|---|
| | conditions | conditions |
| | (YPGa + G418) | (YPD + G418) |
| ADE1 gene disruption | 5.18 | 0.00 |
| efficiency (%) | | |

As is found from the results shown in Tables 2 and 3, a novel method comprising cleaving a nucleic acid fragment containing a gene of interest from a plasmid in a yeast, into which the plasmid has been introduced, using endonuclease (which is homing endonuclease I-SceI in the present example), and then introducing this nucleic acid fragment into the genome according to homologous recombination, could be established. Moreover, the ADE1 gene disruption efficiency was extremely high (approximately 1% to 5%). In other words, the ADE1 gene disruption efficiency calculated in the present example has the same definitions as the efficiency of introducing the nucleic acid fragment cleaved by endonuclease into the genome.

The ADE1 gene disruption efficiency was different between two types of promoters regulating the expression of an endonuclease gene. This was considered because the GAL1 promoter induces expression more strongly than the MET25 promoter. In view of the foregoing, the efficiency of introducing a nucleic acid fragment cleaved by endonuclease into the genome can be further improved by using a stronger promoter as a promoter regulating the expression of the endonuclease gene.

### Example 2

In Example 2, an E. coli strain, NEB Turbo Competent E. coli (NEB), was used as a test line.

Production of Vector to be Introduced into Genome

The vector produced is an E. coli-yeast shuttle vector pRScen-tetR-P_LtetO-SCEI-Ec araB-GFP-SmR-Ec araA comprising the I-SceI gene of the homing endonuclease derived from S. cerevisiae induced by tetracycline (SCEI) and a sequence formed by inserting a DNA fragment comprising homologous recombination sequences to be introduced into the genome between 2 recognition sequences in I-SceI (see FIG. 2). pRScen-tetR-P_LtetO-SCEI-Ec araB-GFP-SmR-Ec araA comprises: the Tet repressor gene derived from transposon Tn10 (NCBI Accession Number: AP000342) (tetR); the SCEI gene linked to the LtetO-1 promoter inducible by tetracycline (Lutz, R. and Bujard, H., “Independent and Tight Regulation of Transcriptional Units in Escherichia coli Via the LacR/O, the TetR/0 and AraC/I1-I2 Regulatory Elements,” Nucleic Acids Research, 25, 1997: 1203-1210); an ampicillin resistance gene; as homologous recombination sequences to be introduced into the genome, the araB gene sequence and the araA gene sequence of the E. coli MG1655 strain (NCBI Accession Number: NC_000913.3); as a gene to be introduced via homologous recombination, a GFP homologous gene (the gene does not comprise a sequence necessary for gene expression such as a promoter sequence and the gene is not expressed; NCBI Accession Number: MI085862); and, as a homologous recombination marker gene, a gene sequence comprising a spectinomycin resistance gene (the smR marker; NCBI Accession Number: No. X12870), inserted into the yeast shuttle vector. This yeast shuttle vector is composed of a region resulting from removal of the GAL1 promoter, the CYC1 terminator, the ADE1 5′ homologous recombination sequence, the G418 marker gene, and the ADE1 3′ homologous recombination sequence from the separately produced vector, pRS436cen(SAT)-P_GAL1-SCEI-T_CYC1-Sce-5U_ADE1-P_AgTEF1-G418-T_AgT EF1-3U_ADE1-Sce.

The pRS436cen(SAT)-P_GAL1-SCEI-T_CYC1-Sce-5U_ADE1-P_AgTEF1-G418-T_AgT EF1-3U_ADE1-Sce vector is a YCp-type yeast shuttle vector in which a 2-microM plasmid-derived replication origin is deleted from pRS436(SAT)-P_GAL1-SCEI-T_CYC1-Sce-5U_ADE1-P_AgTEF1-G418-T_AgTEF1-3U_ADE1-Sce (see Example 1) and, instead thereof, an autonomous replication sequence (ARS) and a centromere sequence (CEN) are inserted therein. The copy number in a cell is retained to be 1 copy.

It should be noted that araB, araA, a GFP homologous gene, and an smR marker sequence are inserted into a region between 2 homing endonuclease I-SceI cleavage recognition sequences, and such region can be cleaved upon expression of the SCEI gene bound to the LtetO-1 promoter induced in a tetracycline-containing medium. A fragment cleaved in an E. coli cell is introduced into the genome via homologous recombination (FIG. 3).

Individual DNA sequences can be amplified by PCR. To bind individual DNA fragments to one another, there were synthesized primers, to each of which a DNA sequence was added to overlap with a DNA sequence adjacent thereto by approximately 15 bp (Table 4). Using these primers, a DNA fragment of interest was amplified with the use of the genome of the MG1655 strain, pRS436cen(SAT)-P_GAL1-SCEI-T_CYC1-Sce-5U_ADE1-P_AgTEF1-G418-T_AgT EF1-3U_ADE1-Sce, the TEF-Dasher GFP plasmid (ATUM), or a synthetic DNA sequence as a template, and the DNA fragments were successively bound to one another using the In-Fusion HD Cloning Kit or the like to produce a final vector of interest.

TABLE 4

| | | SEQ ID |
|---|---|---|
| Amplified DNA fragment | Primer sequence (5′-3′) | NO |
| Fragment containing tetR gene | CATGTTCTTTCCTGCGTTATTAAGACCC | 37 |
| | ACTTTCACATTTAAGTTGTTTTTC | |
| | CTATCACTGATAGGGAGATTTTCACTTT | 38 |
| | TCTCTATCACTGATAGG | |
| Fragment containing LtetO-1 | TCCCTATCAGTGATAGAGATTGACATCC | 39 |
| promoter | CTATCAGTGATAGAGATACTGAGCACA | |
| | TCAGCAGGACGCAC | |
| | AAGTTAAACAAAATTATTTCTAGCTTTC | 40 |
| | TCCTCTITAATGAATTCGGTCAGTGCGT | |
| | CCTGCTGATGTGC | |
| Fragment containing SCE1 gene | GAAATAATTTTGTTTAACTTTAAGAAGG | 41 |
| | AGATATACATAATGAAAAACATTAAGA | |
| | AAAACCAAGTTATO | |
| | TAGGCCAGTACCTCCCGCTTATGTATTT | 42 |
| | ACTCGTAGGATTTGCTTCGTTCGATCAG | |
| | CACAAGCCTTCAAAGATGATCATTTCA | |
| | AGAAGGTTTCGGAGGAG | |
| Fragment containing araB gene | GGGAGGTACTGGCCTAGCGTCGTGGCC | 43 |
| and I-SceI recognition sequence | CGGGAGAGACAGTTTAGTAGTGACTCG | |
| | CGGCCAGTTAGGGATAACAGGGTAATA | |
| | TGGCGATTGCAATTGGC | |
| | CAATCACAGGGGGGGAAATAAGCTACA | 44 |
| | ATTAACOCCAAAAAATTATAGAGTCOC | |
| | AACGGCC | |
| Fragment containing GFP | TCCCGCCCTGTGATTGAGGCGGGATGG | 45 |
| homologous gene | TGTCCCCACAGTATGACCGCACTAACA | |
| | GAAGG | |
| | CATACATTTCTCCACGGGACCCACAGTC | 46 |
| | GTAGATGCGTAAAATCAACCTTGGTAA | |
| | GTATCCAAATCC | |
| Fragment containing smR gene | CGTGGAGAAATGTATGAAACCCTGTAT | 47 |
| | GGAGAGTGATTCAGTCCAGCCAGGACA | |
| | GAAATG | |
| | TTCTCCCAAGTGTACGATATCACACCTA | 48 |
| | GCGCCGTGCAAAAAAAACCACGTCAAA | |
| | TAATCAAGGCGCCTTGAATGCTCGAGG | |
| | GTTATTTGCCGACTACCTTGGTG | |
| Fragment containing araA gene | CGTACACTTGGGAGAAGTCAGATACGA | 49 |
| and I-SceI recognition sequence | TTGCGGCTCAGTATGACGATTTTTGATA | |
| | ATTATGAAGTGTGGTTTG | |
| | CGGCAGTACCGGATCCTAAAGCCGATT | 50 |
| | CAAGAAAAATTACCCTGTTATCCCTATT | |
| | AGCGACGAAACCCGTAATAC | |
| Fragment containing pUC | GGATCCGGTACTGCCGACGCACTTTAG | 51 |
| replication origin, ampicillin | AACGGCCACCGTCCTGGTCCTTTTCATC | |
| resistance gene, autonomously | ACGTGC | |
| replicated sequence (ARS), | TAACGCAGGAAAGAACATGTGAGC | 52 |
| and centromere sequence | | |
| (CEN) | | |

Method of homologous recombination of E. coli genome and verification of introduction efficiency pRScen-tetR-P_LtetO-SCEI-Ec araB-GFP-SmR-Ec araA was transformed into NEB Turbo Competent E. coli, and plasmid-introduced colonies were selected with the aid of ampicillin. Subsequently, the plasmid-introduced colonies were applied on an LB medium containing spectinomycin and a tetracycline analog having the same activity (anhydrotetracycline (50 ng/ml)) to induce homing endonuclease. Thus, a homologous recombination fragment was cleaved from the plasmid. With the use of spectinomycin resistance as the indicator, colonies that had undergone homologous recombination between the homologous recombination fragment and the genome DNA were selected.

In this example, homologous recombination efficiency was calculated by PCR described below. Specifically, a pair or primers indicated with an arrow A and a pair of primers indicated with an arrow B in FIG. 3 were used to perform PCR using genome DNA extracted from the grown colonies as the template (Table 5).

TABLE 5

| Primer | | SEQ |
|---|---|---|
| combination | Primer sequence (5′-3′) | ID NO |
| A | AGCGGATCCTACCTGACGC | 53 |
| | GCGTTACATACCGGATGCG | 54 |
| B | AGCGGATCCTACCTGACGC | 55 |
| | TGGACGGCAGCTGATCCTGCCAGG | 56 |

The pair of primers indicated with an arrow A was designed to amplify a region between the E. coli genome and the homologous recombination fragment. The pair of primers indicated with an arrow B was designed to be positioned on the both sides of the genome to sandwich the homologous recombination fragment. Upon homologous recombination between the E. coli genome and the homologous recombination fragment, DNA fragments of interest were amplified by the pair of primers indicated with an arrow A and the pair of primers indicated with an arrow B. As a result of PCR using the pair of primers indicated with an arrow B, a DNA fragment with the length being increased by the length of the homologous recombination fragment was amplified. With the use of the same as the indicator, homologous recombinants were counted, and homologous recombination efficiency was then calculated.

Homologous Recombination Efficiency of the Vector to be Introduced into E. coli Genome

Concerning the strains into which the pRScen-tetR-P_LtetO-SCEI-Ec araB-GFP-SmR-Ec araA vector had been introduced, many spectinomycin resistant colonies were obtained after homing endonuclease was induced by anhydrotetracycline. Some colonies that had acquired spectinomycin resistance were subjected to PCR in order to inspect whether or not normal homologous recombination had taken place therein. As a result, all the 10 inspected clones were found to have undergone homologous recombination as expected and such clones were found to have undergone very accurate homologous recombination with genome DNA. The method of transformation described in this example can be performed more easily compared with a standard method for homologous recombination of E. coli genome; i.e., the lambda-Red recombination system. By the method of transformation described herein, in addition, homologous recombination efficiency that is sufficient at a practical level has been achieved. Accordingly, it can be said that the method of transformation described herein is a technique with high versatility.

All publications, patents, and patent applications cited herein are incorporated herein by reference in their entirety.

1. A plasmid for transformation, comprising a site into which a gene of interest is to be incorporated, a pair of homologous recombination sequences sandwiching the site, and a pair of endonuclease target sequences sandwiching the pair of homologous recombination sequences.

2. The plasmid for transformation according to claim 1, further comprising a target-specific endonuclease gene specifically cleaving the double strands of the endonuclease target sequences in an expressible state.

3. The plasmid for transformation according to claim 2, wherein the target-specific endonuclease gene is a homing endonuclease gene.

4. The plasmid for transformation according to claim 3, wherein the endonuclease target sequence is specifically recognized by homing endonuclease.

5. The plasmid for transformation according to claim 2, further comprising an inducible promoter regulating the expression of the target-specific endonuclease gene.

6. The plasmid for transformation according to claim 1, which has the gene of interest that is incorporated into the site.

7. A method for producing a transformant, comprising steps of: introducing the plasmid for transformation according to claim 6 into a host; and selecting a transformant, in which the gene of interest comprised in the plasmid for transformation is incorporated into the genome of the host via the homologous recombination sequences comprised in the plasmid for transformation, and the gene of interest is then expressed therein.

8. A transformation method comprising a step of introducing the plasmid for transformation according to claim 6 into a host, wherein the gene of interest comprised in the plasmid for transformation is expressed in the host.

9. The transformation method according to claim 8, wherein the gene of interest is incorporated into the genome of the host via the homologous recombination sequences comprised in the plasmid for transformation.
